(19) United States
(12) Patent Application Publication
(10) Pub. No.: US 2022/0378319 A1
(43) Pub. Date: Dec. 1, 2022

(54) SYSTEMS AND METHODS FOR INSPIRATE SENSING TO DETERMINE A PROBABILITY OF AN EMERGENT PHYSIOLOGICAL STATE

(71) Applicant: GMECI, LLC, Beavercreek, OH (US)

(72) Inventors: Bradford R. Everman, Haddonfield, NJ (US); Brian Scott Bradke, Brookfield, VT (US)

(73) Assignee: GMECI, LLC, Beavercreek, OH (US)

(21) Appl. No.: 17/333,169

(22) Filed: May 28, 2021

(51) Int. Cl. A61B 5/08; G16H 40/67; G16H 50/30; G16H 50/20; G06N 20/00; A61B 5/097; A61B 5/00; A61B 5/091

(52) U.S. Cl. CPC A61B 5/082; G16H 40/67; G16H 50/30; G16H 50/20; G06N 20/00; A61B 5/097; A61B 5/7264; A61B 5/0082; A61B 5/7267; A61B 5/0816; A61B 5/091; A61B 2560/0247

(57) ABSTRACT

Aspects relate to systems and methods for inspirate sensing to determine a probability of an emergent physiological state. An exemplary system an inhalation sensor module configured to sense and transmit a plurality of inhalation parameters as a function of at least an inspirate, an environmental sensor module configured to sense and transmit a plurality of environmental parameters as a function of an environment, and a processor configured to generate a probability of an emergent physiological state by: inputting at least an environmental parameter and at least an inhalation parameter to a probabilistic machine learning model and generating the probability of an emergent physiological state as a function of the machine learning model.

### FIELD OF THE INVENTION

The present invention generally relates to the field of vehicle systems and alarms. In particular, the present invention is directed to systems and methods for inspirate sensing to determine a probability of an emergent physiological state.

### BACKGROUND

Presently, emergent physiological states, such as hypoxia or atelectasis pose a risk to those performing physically challenging tasks, such as flying military aircraft like fighter jets. In these cases a sudden loss of consciousness or impairment of ability may prove catastrophic. Once an emergent physiological state has presented, there is often little that can be done.

### SUMMARY OF THE DISCLOSURE

In an aspect, a system for inspirate sensing to determine a probability of an emergent physiological state includes a fluid channel configured to be in fluidic communication with at least an inspirate, an inhalation sensor module, in fluidic communication with the fluidic channel, configured to sense and transmit a plurality of inhalation parameters as a function of the at least an inspirate, an environmental sensor module, in sensed communication with an environment substantially outside of the fluid channel, configured to sense and transmit a plurality of environmental parameters as a function of the environment, and a processor, in communication with the inhalation sensor module and the environmental sensor module, where the processor is additionally configured to receive the plurality of inhalation parameters and the plurality of environmental parameters, generate a probability of an emergent physiological state, where generating a probability of an emergent physiological state additionally include inputting at least an environmental parameter of the plurality of environmental parameters and at least an inhalation parameter of the plurality of inhalation parameters to a probabilistic machine learning model and generating the probability of an emergent physiological state as a function of the machine learning model.

In another aspect a method of inspirate sensing to determine a probability of an emergent physiological state includes fluidically communicating, using a fluid channel, at least an inspirate, sensing and transmitting, using an inhalation sensor module, a plurality of inhalation parameters as a function of the at least an inspirate, sensing and transmitting, using an environmental sensor module in sensed communication with an environment, a plurality of environmental parameters as a function of the environment, receiving, using a processor in communication with the inhalation sensor module and the environmental sensor module, the plurality of inhalation parameters and the plurality of environmental parameters, generating, using the processor, a probability of an emergent physiological state, wherein generating a probability of an emergent physiological state additionally includes inputting at least an environmental parameter of the plurality of environmental parameters and at least an inhalation parameter of the plurality of inhalation parameters to a probabilistic machine learning model and generating the probability of an emergent physiological state as a function of the machine learning model.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary system for inspirate sensing to determine a probability of an emergent physiological state;

FIG. 2 is a block diagram illustrating an exemplary inhalation sensor module;

FIG. 3 is a block diagram illustrating an exemplary environmental sensor module;

FIG. 4 is a block diagram illustrating an exemplary machine-learning module;

FIG. 5 is a graph representing an exemplary embodiment of a fuzzy set comparison;

FIG. 6 is a flow diagram illustrating an exemplary method of inspirate sensing to determine a probability of an emergent physiological state; and

FIG. 7 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for inspirate sensing. In an embodiment, inspirate sensing may be utilized to predict a probability of an emergent physiological state.

Aspects of the present disclosure can be used to predict a probability of an emergent physiological state and respond with interventions as a function of the probability of occurrence. Aspects of the present disclosure can also be used to warn a user of a probable emergent physiological state. This is so, at least in part, because a user may be able to respond and act to change a probability of an emergent physiological state and prevent the emergent physiological state.

Aspects of the present disclosure allow for a probability of an emergent physiological state, once determined, to be used to intervene for example through communications with a vehicle controller, which may automatically respond to prevent an emergent physiological state. Exemplary embodiments illustrating aspects of the present disclosure are described below in the context of several specific examples.

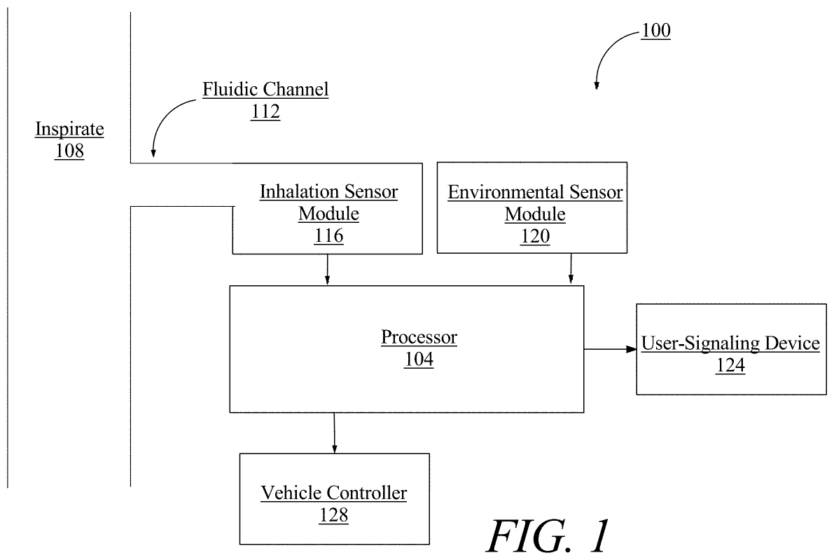

Referring now to FIG. 1, an exemplary embodiment of a system 100 for inspirate sensing is illustrated. System includes a processor 104. Processor 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Processor 104 may include a single computing device operating independently, or may include two or more computing device operating in concert, in parallel, sequentially or the like; two or more computing devices may be included together in a single computing device or in two or more computing devices. Processor 104 may interface or communicate with one or more additional devices as described below in further detail via a network interface device. Network interface device may be utilized for connecting processor 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Processor 104 may include but is not limited to, for example, a computing device or cluster of computing devices in a first location and a second computing device or cluster of computing devices in a second location. Processor 104 may include one or more computing devices dedicated to data storage, security, distribution of traffic for load balancing, and the like. Processor 104 may distribute one or more computing tasks as described below across a plurality of computing devices of computing device, which may operate in parallel, in series, redundantly, or in any other manner used for distribution of tasks or memory between computing devices. Processor 104 may be implemented using a “shared nothing” architecture in which data is cached at the worker, in an embodiment, this may enable scalability of system 100 and/or computing device.

With continued reference to FIG. 1, processor 104 may be designed and/or configured to perform any method, method step, or sequence of method steps in any embodiment described in this disclosure, in any order and with any degree of repetition. For instance, processor 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, as described above system 100 is configured to sense at least an inspirate 108. As used in this disclosure, an “inspirate” is fluid, for example air, belonging to a single inhalation during respiration. System 100 may be configured to sense inspirate to determine a probability of an emergent physiological state. As used in this disclosure, an “emergent physiological state” refers to any human state or condition that arises acutely diminishes performance of one affected. Non-limiting examples of emergent physiological states may include hypoxia, hypocapnia, hypercapnia, hyperventilation, hyperventilation, atelectasis, and the like. Inspirate 108 may be contained for example by a hose and/or tube. For example, in some cases, inspirate 108 may flow within a breathing tube from a fluid source to a mask of a user. In a non-limiting example, user may be an aircraft pilot and mask may be a flight mask.

With continued reference to FIG. 1, system may include a fluid channel 112. As used in this disclosure, a “fluid channel” is any pathway for a flow of fluid; for instance without limitation, a fluid channel may include any of a manifold, a plenum, a hose, a tube, a conduit, and the like. In some cases, fluidic channel 112 may be fluidic communication with at least an inspirate 108. As used in this disclosure, “fluidic communication” is a relationship between two or more things between which fluid, which may include without limitation a liquid or a gas, may pass; as a non-limiting example, a straw provides fluidic communication between a beverage and a drinker's mouth; as another non-limiting example a snorkel provides fluidic communication between an underwater swimmer and air above the water. In some cases, fluidic channel 112 may be configured to direct a portion of at least an inspirate 108, for instance when fluidic channel 112 is branched off from at least an inspirate 108 flow. Alternatively or additionally, in some cases, fluidic channel 112 may be configured to direct substantially all of at least an inspirate 108, for instance when fluidic channel 112 is placed substantially in-line with the at leas an inspirate 108 flow. In some cases, fluidic channel 112 may direct at least an inspirate 108 to an inhalation sensor module 116.

With continued reference to FIG. 1, an inhalation sensor module 116 may be in fluidic communication with fluidic channel 112. As used in this disclosure, an “inhalation sensor module” is a plurality of sensors which are disposed and configured to sense a plurality of inhalation parameters as a function of at least an inspirate. As used in this disclosure, an “inhalation parameter” is a parameter related to inhalation, for example of a user; in some cases, inhalation parameter may include a quantified value or metric. In some cases, inhalation sensor module 116 may be configured to sense and transmit a plurality of inhalation parameters as a function of at least an inspirate 108. In some cases, inhalation sensor module 116 may include a plurality of inhalation sensors. Non-limiting examples of inhalation sensors may include inhalation gas flow sensors, inhalation gas pressure sensors, inhalation gas temperature sensors, inhalation gas humidity sensors, inhalation gas concentration sensors, and the like. Inhalation sensor module 116 may be in communication with processor 104. Communication between inhalation sensor module 116 and processor may include any form of communicative connection including, but not limited to digital, analog, electrical, and/or optical communication. As used herein, a device, component, or circuit is “communicatively connected” where the device, component, or circuit is able to receive data from and/or transmit data to another device, component, or circuit. In an embodiment, devices are placed in communicative connection by electrically coupling at least an output of one device, component, or circuit to at least an input of another device, component, or circuit. Devices may further be placed in communicatively connection by creating an optical, inductive, or other coupling between two or more devices. Communicatively connected device may be placed in near field communication with one another. Two or more devices may be communicatively connected where two or more devices are configured to send and/or receive signals to or from each other. Placement of devices in communicative connection may include direct or indirect connection and/or transmission of data; for instance, two or more devices may be connected or otherwise communicatively connected by way of an intermediate circuit. Placement of devices in communicative connection with each other may be performed via a bus or other facility for intercommunication between elements of processor 104 or a computing device as described in further detail below. Placement of devices in communicative connection with each other may include fabrication together on a shared integrated circuit and/or wafer; for instance, and without limitation, two or more communicatively coupled devices may be combined in a single monolithic unit or module.

With continued reference to FIG. 1, system may additionally include an environmental sensor module 120. Environmental sensor module 120 may be in sensed communication with an environment substantially outside of fluid channel 112. As used in this disclosure, “sensed communication” is an arrangement between two or more relata, in which a first relatum, for example a sensor, is sensibly related to a second relatum, for example an environmental fluid being sensed. In some cases, environmental sensor module 120 may be configured to sense and transmit a plurality of environmental parameters as a function of environment. In some cases, environmental sensor module 120 may include a plurality of environmental sensors. Non-limiting examples of environmental sensors may include environmental gas flow sensors, environmental gas pressure sensors, environmental gas temperature sensors, environmental gas humidity sensors, environmental gas concentration sensors, inertial measurement units (IMUs), and the like. Environmental sensor module 120 may be in communication with processor 104. Communication between environmental sensor module 120 and processor may include any form of communication described in this disclosure.

With continued reference to FIG. 1, processor 104 may be in communication with inhalation sensor module 116 and environmental sensor module 120. In some cases, processor 104 may be configured to receive a plurality of inhalation parameters from inhalation sensor module 116 and receive a plurality of environmental parameters from environmental sensor module 120. In some cases, processor may determine a respiration rate as a function of plurality of inhalation parameters. For example in some cases, an inhalation pressure and/or an inhalation flow may be included in plurality of inhalation parameter; the inhalation pressure and/or inhalation flow may be representative of press and/or flow of at least an inspirate 108 over time. In some cases, processor 104 may determine a respiration rate by processing an inhalation parameter, such as pressure or flow, and determining a number of a extrema within a given time period. For example, in some cases at least one of inhalation pressure or inhalation flow may be derived to find at least one of change in inhalation pressor or flow; the number of times the change in at least one of inhalation pressure or inhalation flow crosses zero over a given time period may be counted; and the number of time the change in at least one of inhalation pressure or inhalation flow crosses zero for a given time period may be divided by time period to determine a respiration rate. In some cases, processor 104 may be configured to determine a respiration volumetric flow rate as a function of plurality of inhalation parameters. In some cases, plurality of inhalation parameters may include a parameter that is representative, or nearly representative, of a respiration volumetric flow rate and processor 104 may simply determine the respiration volumetric flow rate by receiving the parameter that is representative of respiration volumetric flow rate. Alternatively or additionally, in some cases processor 104 may be configured to determine respiration volumetric flow rate from inhalation pressure parameter. In some cases, volumetric flow rate may be derived from one or more fluid dynamic equations, for example Bernoulli's equation. In another exemplary embodiment, volumetric flow rate may be derived using at least one of a Lagrangian and/or a Eulerian fluid mechanics approach.

Referring now to FIG. 1, system 100 may include a sensor, for example as a component within inhalation sensor module 116 and/or environment sensor module 120. Sensor may be configured to detect one or more quantities and/or percentages of gases. In an embodiment, sensor may be configured to detect a carbon dioxide level and generate sensor outputs indicating detected carbon dioxide level. Sensor may alternatively or additionally detect one or more gases, droplets, particulate elements, or the like, which may be indicative of health and/or physiological status of a person using system 100, of environmental conditions that may affect such status, or both. Sensor may be configured to detect a carbon dioxide level by detecting a level of a related compound detecting the carbon dioxide level as a function of the level of the related compound. A “related compound,” as used in this disclosure, is a compound quantities, percentages, and/or concentrations of which may be used to predict quantities, percentages, and/or concentrations of carbon dioxide in one or more contexts, owing to statistical correlations between the two. For instance, and without limitation, quantities, percentages, and/or concentrations of carbon dioxide from sources such as humans and/or other animals may be proportional to quantities, percentages, and/or concentrations of H₂ (hydrogen) gas and/or volatile organic compounds. In an embodiment, a related compound may be more readily or accurately detected using an electrical component of a sensor. Sensor may detect a quantity, percentage, and/or concentration of a related compound such as H₂, volatile organic compounds (VOCs), or the like and calculating an associated level of CO₂ and/or O₂. Sensor may be configured to detect quantities, percentages, and/or concentrations of any other compound directly and/or by detection of a related compound and calculation of the quantities, percentages, and/or concentrations. Such a signal may be used to distinguish the influence of a human presence from other contaminants; for instance, in indoor environments, H₂ concentration may be related to CO₂ concentration as human breath contains significant concentrations of both, CO₂ (4%) and H₂ (10 ppm).

Still referring to FIG. 1, a sensor may be configured to detect quantities, percentages, and/or concentrations of hydrogen gas (H₂). Sensor may be configured to sense quantities, percentages, and/or concentrations of one or more volatile organic compounds. A “volatile organic compound,” as used in this disclosure, are organic compounds having high vapor pressure at room temperature. Volatile organic compounds may include without limitation, alcohols such as ethanol, isoprene, chlorofluorocarbons, benzine, methylene chloride, perchloroethylene, methyl tert-butyl ether (MTBE), and/or formaldehyde. Sensor may be configured to detect a total volatile organic compound (tVOC) quantities, percentage, and/or concentration. “Total volatile organic compound,” as used in this disclosure, is a total concentration of volatile organic compounds present simultaneously in the air. Sensor may detect tVOC using a sensor that is sensitive to sets of volatile organic compounds, a sensor that is sensitive to each of a plurality of volatile organic compounds, and/or sensitive to one or more organic compounds having a quantities, percentages, and/or concentrations of which may be used to predict quantities, percentages, and/or concentrations of tVOC and/or components thereof. For instance, and without limitation, quantities, percentages, and/or concentrations of ethanol in air may be associated with quantities, percentages, and/or concentrations of other volatile organic compounds; sensor may be configured to detect levels and/or quantities of ethanol and calculate tVOC using such detected quantities.

With continued reference to FIG. 1, sensor may alternatively or additionally be configured to sense one or more hazardous gases, droplets, particulate matter or the like, including without limitation hazardous gases, droplets, particulate matter produced by indoor or outdoor air pollution sources, whether natural or anthropogenic, hazardous gases, droplets, particulate matter produced intentionally as an act of violence or war, or the like. Alternatively or additionally, sensor may be configured to detect one or more diagnostically useful gases, droplets, particulate matter or the like, where a “diagnostically useful” gas, droplet, and/or element of particulate matter is defined as a gas, droplet, and/or element of particulate matter that provides information usable to determine a physiological state of a user, for instance as described in further detail below.

Still referring to FIG. 1, a sensor may function using any suitable technology, including without limitation a detector, defined as a circuit element that modifies a circuit parameter when exposed to a compound to be detected. For instance, and without limitation, sensor may use a heating element to temperature of a heated metal-oxide detector, such as a tin-based component that changes resistance based on exposure to a compound to be detected; output may be fed to an operational amplifier, such as without limitation an operational amplifier configured to cover a measurement range of 8 orders of magnitude. Sensor may include, for instance, a first such detector configured to detect CO₂ and/or O₂ and/or a related compound and a second detector configured to detect tVOC and/or a representative compound such as ethanol as described above. Sensor may include, without limitation, input and output ports, a microcontroller for performing calculations as described above, one or more registers and/or more memory elements such as without limitation random-access memory (RAM) such as block random-access memory (BRAM), flash memory, or the like. Sensor may include one or more wireless transceivers or other devices for communication with other elements of system 100, and/or may be wired to such elements. Sensor may be connected to a power source such as a battery or other voltage source.

Alternatively or additionally, and still referring to FIG. 1, sensor may include one or more sensors and/or detectors operating according to one or more additional technologies, such as without limitation at least a chemical sensor, which may be based on polymer or heteropolysiloxane; chemical sensor may be configured to detect concentrations of CO₂ and/or O₂, estimated CO₂ and/or O₂, tVOC, and/or any other element that may be detected by sensor as above.

With continued reference to FIG. 1, in some embodiments, processor 104 may be configured to generate a probability of an emergent physiological state by utilizing machine-learning processes. In some cases, processor 104 may generate a probability of an emergent physiological state by inputting at least an environmental parameter from plurality of environmental parameters and at least an inhalation parameter from plurality of inhalation parameters to a probabilistic machine learning model and generating the probability of an emergent physiological state as a function of the machine learning model. Machine-learning processes are explained in greater detail below. For example, in some cases, processor 104 may be further configured to train a probabilistic machine learning model. In some cases, training probabilistic machine learning model may additionally include inputting training data to a machine learning algorithm, wherein the training data comprises a plurality of parameters correlated to a probabilistic outcome; and training the probabilistic machine learning model as a function of the machine learning algorithm. In some cases, plurality of parameters may include parameters representative of inhalation parameters and/or environmental parameters. For example, in some cases, training data may include inhalation parameter values for one or more of respiration rate, respiration volumetric flow rate, cabin pressure, and/or movement correlated to a deterministic outcome. In some cases, deterministic outcome may represent presence and/or absence of an emergent physiological state. Alternatively or additionally, in some cases, deterministic outcome may include data representing time until presence of an emergent physiological state. In some cases, training data may be user specific; that is to say, in some cases training data may correlate plurality of parameters to deterministic outcomes associated with a specific user. Alternatively, in some cases, training data may be cohort specific or universal and deterministic outcomes are associated with a plurality of users. In some case, a user demographic, for instance cohort identifiers, may be included in training data. Training data may be assembled from historic data. For example, in some embodiments training data may include historic inhalation parameters and previously diagnosed physiological states that have been correlated to the historic inhalation parameters. In cases including supervised machine-learning processes, correlation of historic inhalation parameters and previously diagnosed physiological states may be performed by a user. Alternatively or additionally, historic inhalation parameters may be assembled within a training data set through unsupervised machine-learning processes. In some cases, training data may include theoretical or analytical information in addition or instead of historic data. For example, in some cases, training data may include theoretical ranges and/or thresholds for inhalation parameters which are believed to probabilistically correlate to certain physiological states.

With continued FIG. 1, in some embodiments, processor 104 may be configured to classify a probability of an emergent physiological state to an intervention. In some cases, classifying may additionally include inputting a probability of an emergent physiological state to a classifier; and classifying the probability of an emergent physiological state to the intervention as a function of the classifier. Classifier may include any classifier described in this disclosure. For example, classifier may include any number of frequentist classifiers, Bayesian classifiers, binary and/or multiclass classifiers, vector-based classifiers, linear classifiers, k-nearest neighbor classifiers, and the like. In some cases, processor 104 may be configured to train classifier. In some cases, training classifier may additionally include inputting training data to a machine learning algorithm, wherein the training data comprises a plurality of parameters correlated to candidate interventions; and training the classifier as a function of the machine learning algorithm. In some cases, candidate interventions may include interventions warranted or otherwise desired under certain probabilities of emergent physiological state. In some cases, candidate interventions may be correlated directly, for example by an expert; training of the classifier may be performed according to supervised machine learning processes. Alternatively, in some cases, candidate interventions may be correlated to a plurality of parameters through at least an unsupervised machine learning process.

An intervention may include any response which may be taken as a result of a probability of an emergent physiological state. For example, an intervention may include a response automatically taken by at least an additional system communicative with processor 104. Additionally or alternatively an intervention may include a response that is to be taken by at least a user of the system 100.

Still referring to FIG. 1, in some embodiments, system 100 may include a user-signaling device 124. In some cases, user-signaling device may be communicative with processor 104. In some cases, user-signaling device 124 may be to transmit a signal to a user as a function of intervention from processor 104. Processor 104 may be communicatively connected to at least a user-signaling device 124. In an embodiment, at least a user-signaling device 124 may be incorporated in system 100. Alternatively or additionally, system 100 may communicate with a user-signaling device 124 that is not incorporated in system 100, such as a display, headset, or other device provided by a third party or the like, which may be communicatively connected with processor 104. User-signaling device 124 may be or incorporate a device for communication with an additional user-signaling device 124 such as a vehicle display and/or helmet avionics; for instance, user-signaling device 124 may include a wireless transmitter or transponder communicatively connected with such additional devices.

Continuing to refer to FIG. 1, at least a user-signaling device 124 may include any device capable of transmitting an audible, tactile, or visual signal to a user when triggered to do so by processor 104. In an embodiment, and as a non-limiting example, at least a user-signaling device may include a bone-conducting transducer in vibrational contact with a bone beneath the exterior body surface. A “bone-conducting transducer,” as used herein, is a device or component that converts an electric signal to a vibrational signal that travels through bone placed in contact with the device or component to an inner ear of user, which interprets the vibration as an audible signal. Bone-conducting transducer may include, for instance, a piezoelectric element, which may be similar to the piezoelectric element found in speakers or headphones, which converts an electric signal into vibrations. In an embodiment, bone-conducting transducer may be mounted to housing in a position placing it in contact with a user's bone; for instance, where housing includes or is incorporated in an ear cup, housing may place bone-conducting transducer in contact with user's skull just behind the ear, over the sternocleidomastoid muscle. Likewise, where housing includes a headset, mask, or helmet, housing may place bone-conducting transducer in contact with a portion of user's skull that is adjacent to or covered by headset, mask, or helmet.

Still referring to FIG. 1, at least a user-signaling device 124 may further include an audio output device. Audio output device may include any device that converts an electrical signal into an audible signal, including without limitation speakers, headsets, headphones, or the like. As a non-limiting example, audio output device may include a headset speaker of a headset incorporating or connected to system 100, a speaker in a vehicle user is traveling in, or the like. At least a user-signaling device 124 may include a light output device, which may be any device that converts an electrical signal into visible light; light output device may include one or more light sources such as LEDs, as well as a display, which may be any display as described below. At least a user-signaling device 124 may include a vehicular display; at least a vehicular display may be any display or combination of displays presenting information to a user of a vehicle user is operating. For instance, at least a vehicular display may include any combination of audio output devices, light output devices, display screens, and the like in an aircraft flight console, a car dashboard, a boat dashboard or console, or the like; processor 104 may be communicatively connected with vehicular display using any form of communicative connection described above, including without limitation wired or wireless connection. At least a user-signaling device 124 may include a helmet display; helmet display may include any visual, audio, or tactile display incorporated in any kind of helmet or headgear, which may be communicatively connected with processor 104 according to any form of communicative connection as described above.

Further referring to FIG. 1, any of the above user-signaling device 124 and/or signals may be used singly or in combination; for instance, a signal to user may include an audio signal produced using a bone-conducting transducer, a verbal message output by an audio output device, and a visual display of an image or text indicating an output. Persons skilled in the art, upon reviewing the entirety of this disclosure, will be aware of various combinations of signaling means and/or processes that may be employed to convey a signal to user. In an embodiment, in addition or alternatively to transmitting a signal to user-signaling device 124, processor 104 may transmit a signal to one or more automated vehicular controls or other systems, for instance to alleviate one or more environmental parameters contributing to a probability of an emergent physiological state.

Still referring to FIG. 1, in some embodiments, system may include a vehicle controller 128. Vehicle controller 128 may be communicative with processor 104. In some cases, vehicle controller 128 may be configured to respond as a function of an intervention from processor 104. As a non-limiting example vehicle controller 128 may include a flight control system on an aircraft; and the flight control system may be configured to control the aircraft at least in part based upon intervention. For example, if processor 104 has determined that a pilot is very likely (e.g., greater than 20%, greater than 50%, greater than 75%, or greater than 90%) likely to experience an emergent physiological state, the processor 104 may classify an intervention that includes a flight controller to make a change to flight controls, such as without limitation enable autopilot. Alternatively or additionally, in some cases, processor 104 may classify an intervention that commands a flight controller to change a flight characteristic to decrease a probability of an emergent physiological state, for example by reducing altitude or limiting G-forces.

Still referring to FIG. 1, in some cases, processor 104 may be additionally configured to generate at least a confidence metric. As used in this disclosure, a “confidence metric,” is a quantitative measure of confidence associated with a classification of an intervention. In some cases, a confidence metric may quantify confidence of associated with a classification of an intervention of an intervention from a probability of an emergent physiological state, for example without limitation “processor is 95% confident that engaging autopilot is a warranted intervention to a 90% probability of user to experience hypoxia.” Alternatively, confidence metric may quantify a confidence associated with a probability of an emergent physiological state, for example without limitation “user is 90% probable to experience hypoxia, with a confidence of 95%.” Furthermore in some cases, confidence metric may quantify confidence in probability determinations and classification, for example “engaging autopilot is currently warranted, confidence of 95%.” In some cases, generation of a confidence metric may include aggregation, for example addition and/or multiplication, of one or more sub-confidence metrics. In some cases, generation of a confidence metric additionally includes inputting intervention class to a machine learning model; and generating the confidence metric as a function of the machine learning model. In some cases, generation of a confidence metric may include fuzzy-set classification.

Still referring to FIG. 1, according to some embodiments, system 100 may be incorporated with one or more additional systems including sensors and processors. For example, system 100 may be employed with an earcup system as taught by U.S. patent application Ser. No. 16/859,483 entitled “HUMAN PERFORMANCE OXYGEN SENSOR,” by B. Everman et al., which is incorporated herein by reference, in its entirety. Additionally or alternatively, in some embodiments, system 100 may be employed with an exhalation sensor system as taught by U.S. patent application Ser. No. 16/933,680 entitled “COMBINED EXHALED AIR AND ENVIRONMENTAL GAS SENSOR APPARATUS,” by B. Everman et al., which is incorporated herein by reference, in its entirety. In some embodiments, environmental sensor module 120 may include a combined exhaled air and environmental gas sensor. System 100 may compare exhaled air and environmental gas to inspirate, as measured by inhalation sensor module 116. For example, in some cases, system 100 may detect a leak within an inhalation fluidic channel by comparing at least one of exhalation air and environmental gas to inspirate.

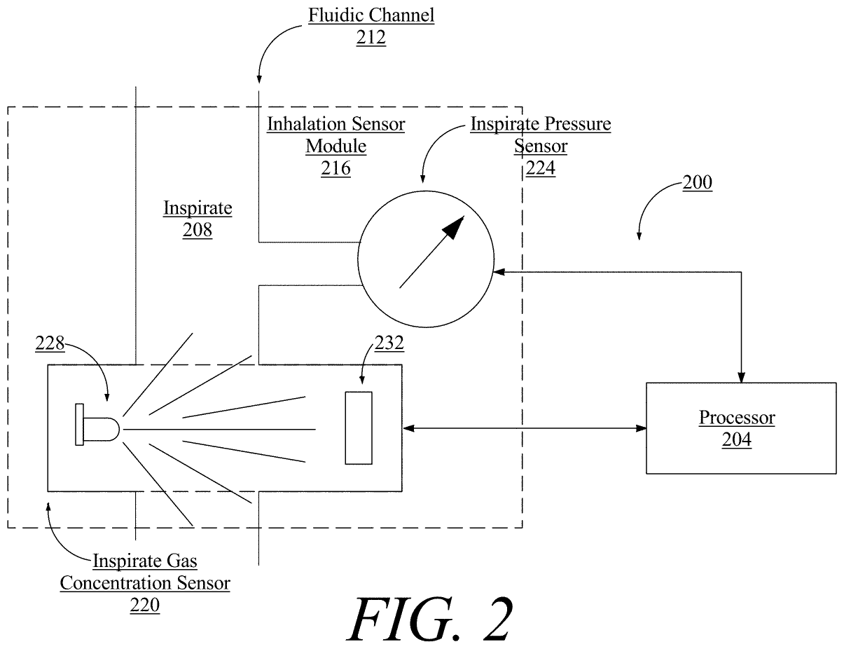

Referring now to FIG. 2, an exemplary block diagram 200 illustrates a processor 204 for making determinations as a function of sensed parameters associated with at least an inspirate 208. in communication with an exemplary inhalation sensor module 208. In some cases, at least a portion of an at least an inspirate 208 is contained within a fluidic channel 212. An exemplary inhalation sensor module 216 is shown in fluid communication with fluidic channel 212. In some cases, inhalation sensor module may include at least a gas concentration sensor 220. In some cases, inhalation sensor module 216 may include at least an inspirate pressure sensor 224. Inspirate gas concentration sensor 220 may include any gas concentration sensor, for instance those described in this application. In some cases, inspirate gas concentration sensor 220 may include an optical gas concentration sensor. Non-limiting optical gas concentration sensors include infrared transmission and/or absorbance spectroscopy type sensors and fluorescence excitation type sensors. Commonly, an optical gas concentration sensor may include a radiation source 228 and a radiation detector 232. In some versions, radiation source 228 may include a light source 228 that may generate a light and illuminate at least a portion of at least an inspirate 208. Radiation source 228 may generate any of a non-limiting list of lights, including coherent light, non-coherent light, narrowband light, broadband light, pulsed light, continuous wave light, pseudo continuous wave light, ultraviolet light, visible light, and infrared light. In some cases, radiation source 228 may include an electromagnetic radiation source that may generate an electromagnetic radiation and irradiate at least a portion of at least an inspirate 208. Radiation source 228 may generate any of a non-limiting list of radiations including radio waves, microwaves, infrared radiation, optical radiation, ultraviolet radiation, X-rays, gamma-rays, and light. Non-limiting examples of radiation sources 228 include lasers, light emitting diodes (LEDs), light emitting capacitors (LECs), flash lamps, antennas, and the like. In some cases, radiation detector 232 may be configured to detect light and/or radiation that has interacted directly or indirectly with at least a portion of at least an inspirate 208. Non-limiting examples of radiation detectors 232 include photodiodes, photodetectors, thermopiles, pyrolytic detectors, antennas, and the like. In some cases, a radiation amount detected by radiation detector 232 may be indicative of a concentration of a particular gas in at least a portion of at least an inspirate 208. For example, in some exemplary embodiments, radiation source 228 may include an infrared light source operating at a wavelength about 4.6 μm and radiation detector may include a photodiode sensitive over a range encompassing 4.6 μm. An exemplary infrared light source may include an LED comprising InAsSb/InAsSbP heterostructures, for example LED46 from Independent Business Scientific Group (IBSG) of Saint Petersburg, Russia. An exemplary infrared detector may include a mercury cadmium telluride photodiode, for example UM-I-6 HgCdTe from Boston Electronics of Brookline, Mass. In some cases, an amount of radiation at at least a specific wavelength absorbed, scatter, attenuated, and/or transmitted may be indicative of a gas concentration.

With continued reference to FIG. 2, in some cases, inspirate concentration sensor 220 may include an infrared point sensor. An infrared (IR) point sensor may use radiation passing through a known volume of gas, for example at least an inspirate 208. In some cases, detector 232 may be configured to detect radiation after passing through gas at a specific spectrum. As energy from infrared may be absorbed at certain wavelengths, depending on properties of at least an inspirate 220. For example, carbon monoxide absorbs wavelengths of about 4.2-4.5 μm. In some cases, detected radiation within a wavelength range (e.g., absorption range) may be compared to a wavelength outside of the wavelength range. A difference in detected radiation between these two wavelength ranges may be found to be proportional to a concentration of gas present. In some embodiments, an infrared image sensors may be used for active and/or passive imaging. For active sensing, radiation source 228 may include a coherent light source (e.g., laser) which may be scanned across a field of view of a scene and radiation detector 232 may be configured to detect backscattered light at an absorption wavelength of a specific target gas. In some cases, radiation detector 232 may include an image sensor, for example a two-dimensional array of radiation sensitive devices, for example arranged as pixels. Passive IR imaging sensors may measure spectral changes at each pixel in an image and look for specific spectral signatures that indicate presence and/or concentration of target gases.

With continued reference to FIG. 2, in some cases, inspirate gas concentration sensor 220 may include an oxygen sensor. An exemplary oxygen sensor may include an electro-galvanic sensor. For example, an electro-galvanic oxygen sensor may be used to measure a concentration of oxygen within at least an inspirate 208. In some cases, an electro-galvanic oxygen sensor may include a lead/oxygen galvanic cell, within which oxygen molecules are dissociated and reduced to hydroxyl ions at a cathode. Hydroxyl ions may diffuse through an electrolyte and oxidize a lead anode. A current proportional to a rate of oxygen consumption may be generated when cathode and anode are electrically connected through a resistor. Current may be sensed by known current sensing methods, for example without limitation those described in this disclosure, to produce an electrical signal proportional to a concentration of oxygen, for example oxygen within at least an inspirate. Another exemplary oxygen sensor may include a lambda sensor, for example a zirconia sensor, a wideband zirconia sensor, and/or a titania sensor. A lambda sensor may be configured to sense a quantity of oxygen in a gas (e.g., at least an inspirate 208) relative another gas, for example air within an environment (e.g., cabin air) and transmit an analog voltage correlated to the sensed relative quantity of oxygen. Analog voltage transmitted by a lambda sensor may be processed by any data or signal processing methods discussed herein, for example through amplification and/or analog-to-digital conversion.

In another exemplary embodiment, inspirate concentration sensor 220 may include an optical sensor configured to sense oxygen concentration. In some cases, a chemical film is configured to be in contact with a gas (e.g., at least an inspirate 208). Chemical film may have fluorescence properties which are dependent upon presence and/or concentration of oxygen. Radiation detector 232 may be positioned and configured, such that it is in sensed communication with chemical film. Radiation source 228 may irradiate and/or illuminate chemical film with radiation and/or light having properties (e.g., wavelength, energy, pulse duration, and the like) consistent with exciting fluorescence within the chemical film. In some cases, fluorescence may be at a maximum when there is no oxygen present. For example, oxygen molecules may collide with chemical film and quench photoluminescence resulting from fluorescent excitation. A number of O₂ molecules colliding with chemical film may be correlated with a concentration of oxygen within a gas (e.g., inspirate 208). Fluorescence properties as sensed by optical detector 232 may therefore be related to oxygen concentration. Fluorescence properties may include emission duration, fluorescence energy, and the like. In some cases, detected optical signal (fluorescence) to oxygen concentration may not be linear. For instance, an optical oxygen sensor may be most sensitive at low oxygen concentration; that is, sensitivity decreases as oxygen concentration increases, following a known Stern—Volmer relationship. In some cases, an optical oxygen sensor is advantageous as substantially no oxygen may be consumed, during sensing. In some cases, planar optical oxygen sensors (i.e., optodes) may be used to detect a spatial distribution of oxygen concentrations over an area, for example as a two-dimensional image. Based on the same principle, radiation detector 232 may include a digital camera that may be used to capture fluorescence intensities over a specific area.

With continued reference to FIG. 2, inhalation sensor module 216 may include at least an inspirate pressure sensor 224, which is fluidic communication with at least an inspirate 208, for example by way of at least a fluidic channel 212. In some cases, at least an inspirate pressure sensor 216 may be configured to sense and transmit at least an inspirate pressure parameter as a function of a pressure of at least an inspirate 208. In some cases, inhalation pressure sensor 224 may include any type of pressure sensor described in this disclosure. Inhalation pressure sensor 224 may be a force collector type pressure sensor. Alternatively, in some case, inhalation pressure sensor 224 may be a pressure sensor type that does not use force collection.

Still referring to FIG. 1, processor 104 may be configured to calibrate at least a sensor to a calibration setting. Calibration setting may be a setting used to calibrate at least a sensor to known concentrations and/or quantities of CO₂ and/or O₂, related compounds, volatile organic compounds, tVOC, or other substances to be sensed by sensor 108. Known concentrations and/or quantities may be determined beforehand, for instance in a laboratory or test facility setting, a sensor that has already been calibrated and/or a sensor having a high accuracy level such as a light spectroscopy sensor, such as a nondispersive infrared (NDIR), in which CO₂ and/or O₂ or other substance of and/or containing gas is pumped or diffused into a light tube, interference (wavelength) filter and light detector such as an infrared detector, and absorption characteristics are measured by dips in transmitted light of one or more frequencies. Calibration relationships between known levels of compounds to be sensed and corresponding sensor outputs may be stored in memory of processor 104, which may implement, for instance and without limitation, a look up table to determine levels of sensed substances based on sensor outputs.

In an embodiment, and with further reference to FIG. 1, processor 104 may be configured to select the calibration setting from a plurality of candidate calibration settings as a function of at least an environmental parameter. At least an environmental parameter may be any parameter and/or combination of parameters detectable by an environmental sensor for instance in environmental sensor module 120, as described above. At least an environmental parameter may include one or more parameters that can affect sensor readings, such as humidity, temperature, and/or air pressure. Sensor may self-correct for variation in such environmental parameters if the environmental parameters are input to sensor; alternatively or additionally, processor 104 may use different calibration settings for different levels of environmental parameters and/or combinations thereof; such levels may be set in a test facility and/or measured in combination with sensor outputs and known levels of substances to be sensed. Processor 104 may compare at least an environmental parameter to one or more values and/or ranges of values to determine which calibration setting to use. Thus, for instance, a first calibration setting may be used for a pressure of approximately 1 atm, a relative humidity level of 30%, and a temperature of 24 degrees Celsius, while a second calibration setting may be used for a pressure of approximately 0.5 atm, a relative humidity level of 10%, and a temperature of 5 degrees Celsius; persons skilled in the art, upon reviewing the entirety of this disclosure, will be aware of various variations in environmental parameters and calibration settings that may be employed.

Alternatively or additionally, and still referring to FIG. 1, processor 104 may use a calibration function of environmental parameters and sensor outputs to determine levels of one or more sensed substances. Calibration function, which may have a form of a linear and/or polynomial function of environmental parameters and sensor outputs, may enable processor 104 to determine accurate concentrations of sensed substances across a continuous range of environmental parameter and sensor output values. In an embodiment, training data may be developed using one or more recorded calibration settings as described above, and then used to train a machine-learning model including a calibration function; training may be performed using a regression algorithm and produce a regression model including calibration function. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 1, machine-learning algorithms used to produce calibration function may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression.

With continued reference to FIG. 2, pressure sensor may include a force collector type pressure sensor. A force collector type pressure sensor collects force over an area, for example by way of a diaphragm, piston bourdon tube, and/or bellows. In some cases, a force collector type pressure sensor may measure strain (i.e., deflection) resulting from collected force. Measured strain may be sued to determine force, for example through Hook's Law; and pressure may be calculated by dividing pressure by area (for example a cross sectional area of force collector). Exemplary non-limiting force collector type pressure sensors may include any number of force transducers, including force transducers using piezoresistance, capacitance, electromagnetism, piezoelectric effect, impedance, optics, potentiometrics, and force balancing. A pressure sensor may include a piezoresistive strain gauge. Piezoresistive effect of bonded or formed strain gauges may be used to detect strain due to applied pressure, with resistance increasing as pressure deforms the material. Common technology types for piezoresistive stain gauges may include Silicon (Monocrystalline), Polysilicon Thin Film, Bonded Metal Foil, Thick Film, Silicon-on-Sapphire and Sputtered Thin Film. A pressure sensor may include a capacitive sensor. A capacitive pressure sensor may use a diaphragm and pressure cavity to create a variable capacitor, which may be used to detect strain due to applied pressure, with capacitance decreasing as pressure deforms the diaphragm. A capacitive pressure sensor may include a metal, ceramic, and/or silicon diaphragm. Pressure sensor may include an electromagnetic sensor. An electromagnetic sensor may measure displacement of a diaphragm by means of changes in inductance (reluctance), linear variable differential transformer (LVDT), Hall Effect, or by eddy current principle. A pressure sensor may be a piezoelectric sensor. A piezoelectric pressure sensor may use piezoelectric effect in certain materials, such as without limitation quartz, to measure a strain upon a sensing mechanism due to pressure. A piezoelectric pressure sensor may be employed for measurement of highly dynamic pressures. In some cases, pressure sensor may include a strain-gauge. Strain gauge-based pressure sensors may include a metal strain gauge adhered to a surface. Alternatively or additionally, in some cases, strain gauge-based pressure sensors may include thin film or be applied on by sputtering. Strain gauge may be measuring strain of a measuring element, such as without limitation a diaphragm. In some cases, pressure sensor may be optical. Optical pressure sensor techniques may include detection of physical change of an optical fiber to detect strain due to applied pressure. An exemplary optical pressure sensor may utilize Fiber Bragg Gratings. In some cases, optical pressure sensors may be used where measurement may be highly remote, under high temperature, or may benefit from technologies inherently immune to electromagnetic interference. In some cases, pressure sensor may be potentiometric and use a motion of a wiper along a resistive mechanism to detect the strain caused by applied pressure. In some additional cases, pressure sensor may utilize force balancing. For example, a force-balanced fused quartz bourdon tubes use a spiral bourdon tube to exert force on a pivoting armature containing a mirror. Reflection of a beam of light from mirror senses an angular displacement and current may be applied to electromagnets on armature to balance force from the tube and bring the angular displacement to zero. Current applied may be used as measurement of pressure. Due to the extremely stable and repeatable mechanical and thermal properties of fused quartz and the force balancing which eliminates most non-linear effects these sensors may be accurate to around 1 PPM of full scale. In some cases, a pressure sensor comprising a strain gauge, or another analog transducer, may be connected to form a Wheatstone bridge circuit to maximize output and reduce sensitivity to errors.

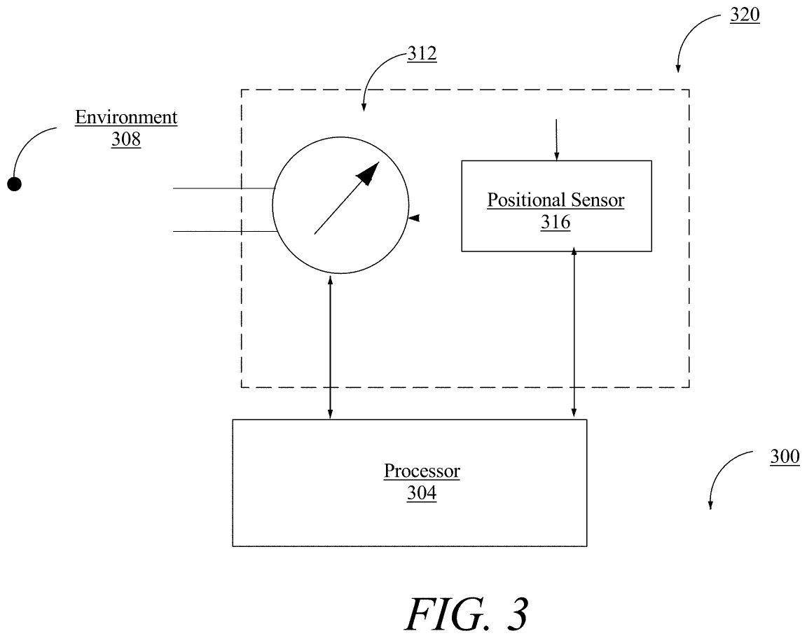

Referring now to FIG. 3, an exemplary block diagram 300 illustrates a processor 304 for making determinations as a function of sensed parameters associated with at least an environment 308. In some cases, environment may be located substantially outside of fluidic channel. For example, in some cases, an environment may substantially include some or all of an interior of a cabin, for example within a cockpit of an aircraft. Additionally or alternatively, environment 308 may include a space proximal a user, for example a volume between a mask and a user's face. In some cases, processor 304 may communicate with at least a cabin pressure sensor 312 and/or a positional sensor 316. In some cases, one or both of cabin pressure sensor 312 and/or positional sensor 316 may be included within an environmental sensor module 320. In some cases, at least a cabin pressure sensor 312 may be configured to sense and transmit at least a cabin pressure parameter as a function of a pressure of environment 308. Cabin pressure sensor 308 may include any pressure sensor described in this disclosure, for example above. In some cases, at least a positional sensor 316 may be configured to sense and transmit at least a movement parameter as a function of a movement. Environmental sensor module 320 may include at least an environmental sensor. An environmental sensor may be any sensor configured to detect at least an environmental parameter. An environmental parameter may include a parameter describing non-physiological data concerning user or surroundings of user. At least an environmental sensor may include at least a positional sensor 316, including without limitation one or more accelerometers, gyroscopes, magnetometers, or the like; at least a positional sensor 316 may include an inertial measurement unit (IMU). At least an environmental sensor 412 may include at least a temperature sensor 416. At least an environmental sensor may include at least a barometric sensor. Environmental sensor module 320 may include a pressure sensor 312, for instance to detect air or water pressure external to user. At least an environmental sensor may include a humidity and/or relative humidity sensor 424.

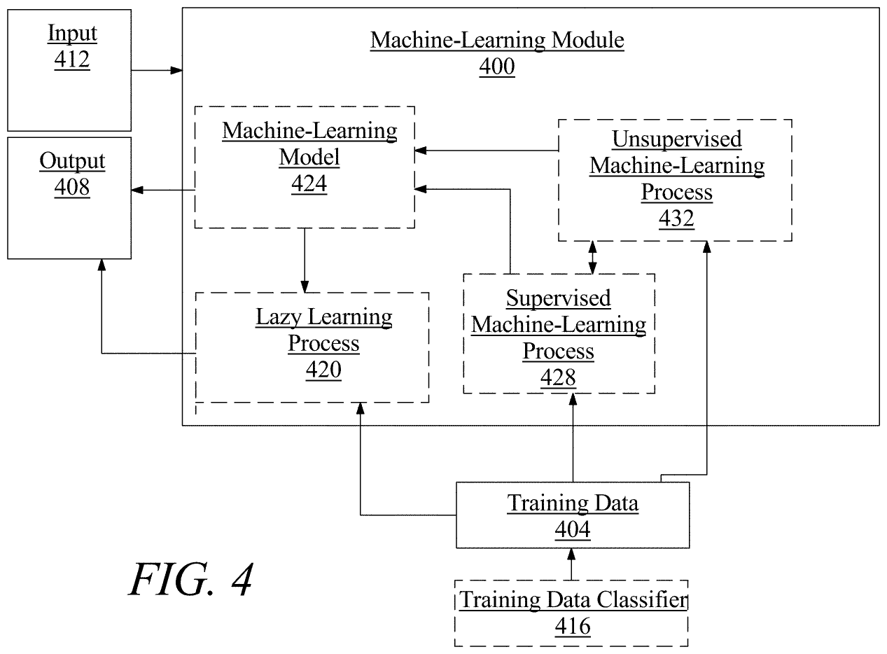

Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example input data may include at least an inhalation parameter and/or at least an environmental parameter and output data may include a deterministic outcome, such as presentation or lack thereof of an emergent physiological state.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data according to individual user, cohort of users or user demographics. In some cases, for example a probability of an emergent physiological state may be generated using training data specific to particular user. Alternatively or additionally, training data may include primarily inhalation parameters and be classified according to environmental parameters. For example, in some cases, a differently trained machine-learning model will be employed under a first set of environmental conditions (e.g., high altitude or high acceleration) than under a second set of environmental conditions (e.g., ordinary flight conditions).

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 4, machine-learning processes as described in this disclosure may be used to generate machine-learning models 424. A “machine-learning model,” as used in this disclosure, is a mathematical and/or algorithmic representation of a relationship between inputs and outputs, as generated using any machine-learning process including without limitation any process, as described above, and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include a probability of an emergent physiological state as described above as inputs, and interventions as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

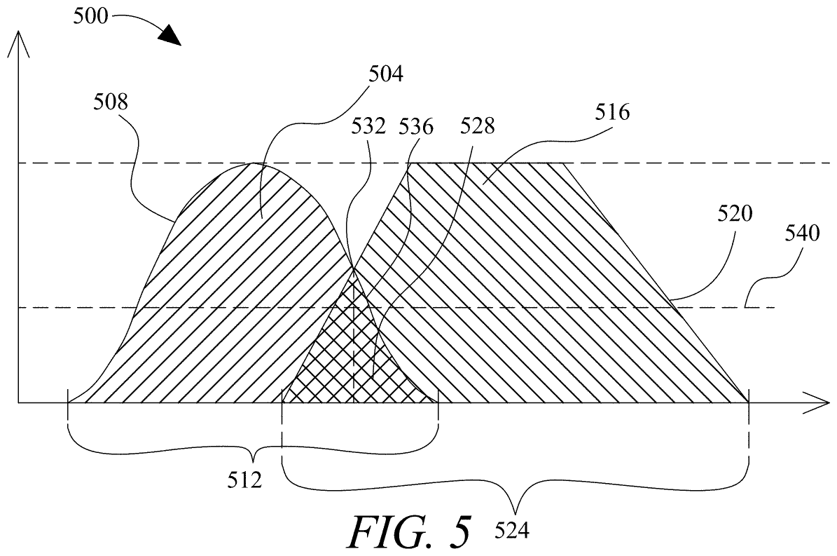

Referring to FIG. 5, an exemplary embodiment of fuzzy set comparison 500 is illustrated. A first fuzzy set 504 may be represented, without limitation, according to a first membership function 508 representing a probability that an input falling on a first range of values 512 is a member of the first fuzzy set 504, where the first membership function 508 has values on a range of probabilities such as without limitation the interval [0,1], and an area beneath the first membership function 508 may represent a set of values within first fuzzy set 504. Although first range of values 512 is illustrated for clarity in this exemplary depiction as a range on a single number line or axis, first range of values 512 may be defined on two or more dimensions, representing, for instance, a Cartesian product between a plurality of ranges, curves, axes, spaces, dimensions, or the like. First membership function 508 may include any suitable function mapping first range 512 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( x , a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- a ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 5, first fuzzy set 504 may represent any value or combination of values as described above, including a probability of an emergent physiological state, an intervention, such as without limitation signaling a user, and/or any combination of the above. A second fuzzy set 516, which may represent any value which may be represented by first fuzzy set 504, may be defined by a second membership function 520 on a second range 524; second range 524 may be identical and/or overlap with first range 512 and/or may be combined with first range via Cartesian product or the like to generate a mapping permitting evaluation overlap of first fuzzy set 504 and second fuzzy set 516. Where first fuzzy set 504 and second fuzzy set 516 have a region 528 that overlaps, first membership function 508 and second membership function 520 may intersect at a point 532 representing a probability, as defined on probability interval, of a match between first fuzzy set 504 and second fuzzy set 516. Alternatively or additionally, a single value of first and/or second fuzzy set may be located at a locus 536 on first range 512 and/or second range 524, where a probability of membership may be taken by evaluation of first membership function 508 and/or second membership function 520 at that range point. A probability at 528 and/or 532 may be compared to a threshold 540 to determine whether a positive match is indicated. Threshold 540 may, in a non-limiting example, represent a degree of match between first fuzzy set 504 and second fuzzy set 516, and/or single values therein with each other or with either set, which is sufficient for purposes of the matching process; for instance, threshold may indicate a sufficient degree of overlap between a probability of an emergent physiological state and an intervention for combination to occur as described above. Alternatively or additionally, each threshold may be tuned by a machine-learning and/or statistical process, for instance and without limitation as described in further detail below.

Further referring to FIG. 5, in an embodiment, a degree of match between fuzzy sets may be used to classify a probability of an emergent physiological state relating to a candidate intervention. For instance, if a probability of an emergent physiological state has a fuzzy set matching an intervention fuzzy set by having a degree of overlap exceeding a threshold, processor 104 may classify the probability of an emergent physiological state as belonging to the intervention. Wherein an intervention is classified, processor 104 may implement intervention by any method described in this disclosure, such as without limitation signaling user or altering control of vehicle. Where multiple fuzzy matches are performed, degrees of match for each respective fuzzy set may be computed and aggregated through, for instance, addition, averaging, or the like, to determine an overall degree of match. In some cases, an overall degree of match may represent a confidence metric; for example a confidence metric indicative of an interventions match to a probability of an emergent physiological state.

Still referring to FIG. 5, in an embodiment, a probability of an emergent physiological state may be compared to multiple predetermined fuzzy sets. For instance, probability of an emergent physiological state may be represented by a fuzzy set that is compared to each of the multiple predetermined class fuzzy sets; and, a degree of overlap exceeding a threshold between the probability fuzzy set and any of the multiple predetermined class fuzzy sets may cause processor 104 to classify the candidate intervention representation as belonging to an intervention, or said another way, as warranting an intervention. For instance, in one embodiment there may be two predetermined class fuzzy sets, representing respectively a non-intervention class and an intervention class. Intervention class may have an intervention class fuzzy set; non-intervention may have a non-intervention class fuzzy set; and probability of an emergent physiological state may have a probability fuzzy set. Processor 104, for example, may compare a probability fuzzy set with each of intervention class fuzzy set and non-intervention class fuzzy set, as described above, and classify a probability of an emergent physiological state to either, both, or neither of intervention class or non-intervention class. Machine-learning methods as described above may, in a non-limiting example, generate coefficients used in fuzzy set equations as described above, such as without limitation x, c, and ci of a Gaussian set as described above, as outputs of machine-learning methods.

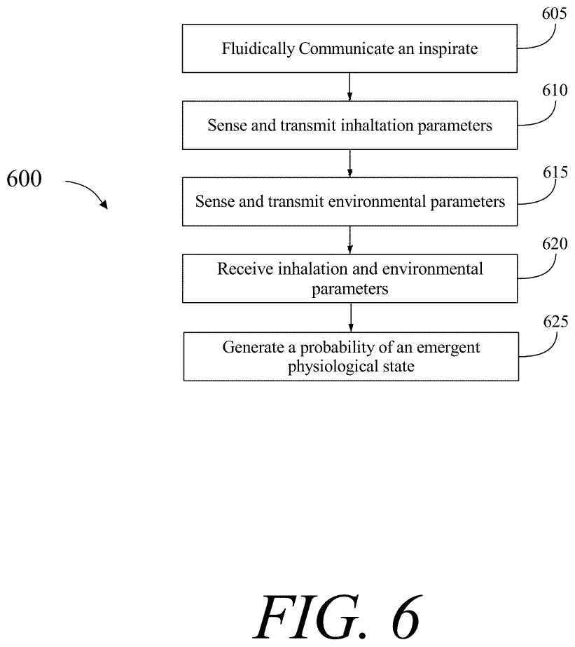

Referring now to FIG. 6, a flow diagram illustrates an exemplary method 600 of inspirate sensing to determine a probability of an emergent physiological state. At step 605, a fluid channel may fluidically communicate at least an inspirate. A fluid channel may include any fluid channel described in this disclosure, for example in reference to FIGS. 1-5. Fluidically communicating may include any method of fluidic communication described in this disclosure, for example in reference to FIGS. 1-5. At least an inspirate may include any inspirate described in this disclosure, for example in reference to FIGS. 1-5.

Continuing in reference to FIG. 6, at step 610, an inhalation sensor module may sense and transmit a plurality of inhalation parameters as a function of at least an inspirate. Inhalation sensor module may include a plurality of any sensors described in this disclosure, for example in reference to FIGS. 1-5. Plurality of inhalation parameters may include any inhalation parameters described in this disclosure, for example in reference to FIGS. 1-5. In some embodiments, step 610 may additionally include, a gas concentration sensor sensing and transmitting at least an inspirate gas concentration parameter as a function of a gas concentration within at least an inspirate. Gas concentration sensor may include any gas concentration sensor described in this disclosure, for example in reference to FIGS. 1-5. In some cases, sensing and transmitting at least an inspirate gas concentration parameter may additionally include illuminating, using a light source, a portion of at least an inspirate, and detecting, using a light detector, a light, wherein the light is either originated from the light source or excited by light from the light source. In some embodiments, step 610 may additionally include, an inspirate pressure sensor sensing and transmitting at least an inspirate pressure parameter as a function of a pressure of at least an inspirate. Inspirate pressure sensor may include any pressure sensor described in this disclosure, for example in reference to FIGS. 1-5.

Continuing in reference to FIG. 6, at step 615, an environmental sensor module may sense and transmit a plurality of environmental parameters as a function of an environment. Environmental sensor module may include a plurality of any sensors described in this disclosure, for example in reference to FIGS. 1-5. Plurality of environmental parameters may include any environmental parameters described in this disclosure, for example in reference to FIGS. 1-5. Environment may include any environment as described in this disclosure, for example in reference to FIGS. 1-5. In some embodiments, step 615 may additionally include sensing and transmitting, using at least a cabin pressure sensor, at least a cabin pressure parameter as a function of a pressure of environment, and sensing and transmitting, using at least a positional sensor, at least a movement parameter as a function of a movement. Cabin pressure sensor may include any pressure sensor described in this disclosure, for example in reference to FIGS. 1-5. Positional sensor may include any positional sensor described in this disclosure, for example in reference to FIGS. 1-5.

Continuing in reference to FIG. 6, at step 620, a processor may receive a plurality of inhalation parameters and a plurality of environmental parameters. Processor may include any processor and/or computing device as described in this disclosure, for example in reference to FIGS. 1-5.

Continuing in reference to FIG. 6, at step 625, processor may generate a probability of an emergent physiological state. Probability of an emergent physiological state may include any probability of an emergent physiological state as described in this disclosure, for example in reference to FIGS. 1-5. In some cases, step 625 may additionally include: inputting at least an environmental parameter of plurality of environmental parameters and at least an inhalation parameter of plurality of inhalation parameters to a probabilistic machine learning model; and generating probability of an emergent physiological state as a function of the machine learning model.

Still referring to FIG. 6, in some embodiments, method 600 may additionally include classifying, using processor, probability of an emergent physiological state to an intervention. Intervention may include any intervention described in this disclosure, for example in reference to FIGS. 1-5. In some cases, classifying probability of an emergent physiological state to intervention may include inputting the probability of an emergent physiological state to a classifier and classifying the probability of an emergent physiological state to the intervention as a function of the classifier. Classifier may include any classifier as described in this disclosure, for example in reference to FIGS. 1-5. In some cases, method may additionally include training, using processor, classifier. Training classifier may additionally include inputting training data to a machine learning algorithm, wherein the training data comprises a plurality of parameters, and training the classifier as a function of the machine learning algorithm.

Still referring to FIG. 6, in some embodiments, method 600 may additionally include transmitting, using a user-signaling device communicative with processor, a signal to a user as a function of the intervention. User-signaling device may include any user-signaling device as described in this disclosure, for example in reference to FIGS. 1-5. In some cases, method may additionally include generating, using processor, at least a confidence metric. Confidence metric may include any confidence metric as described in this disclosure, for example in reference to FIGS. 1-5. In some cases, generating at least a confidence metric additionally includes inputting intervention class to a machine learning model and generating the at least a confidence metric as a function of the machine learning model.

Still referring to FIG. 6, in some embodiments, method 600 may additionally include training, using processor, probabilistic machine learning model. In some cases, training probabilistic machine learning model may additionally include inputting training data to a machine learning algorithm, wherein the training data comprises a plurality of parameters correlated to a probabilistic outcome, and training the probabilistic machine learning model as a function of the machine learning algorithm. Training data may include any training data described in this disclosure, for example in reference to FIGS. 1-5. Machine learning algorithm may include any machine learning algorithm described in this disclosure, for example in reference to FIGS. 1-5.

Still referring to FIG. 6, in some embodiments, method 600 additionally includes determining, using processor, a respiration rate as a function of plurality of inhalation parameters and determining, using the processor, a respiration volumetric flow rate as a function of the plurality of inhalation parameters. Respiration rate may include any respiration rate as described in this disclosure, for example in reference to FIGS. 1-5. Respiration volumetric flow rate may include any flow rate as described in this disclosure.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

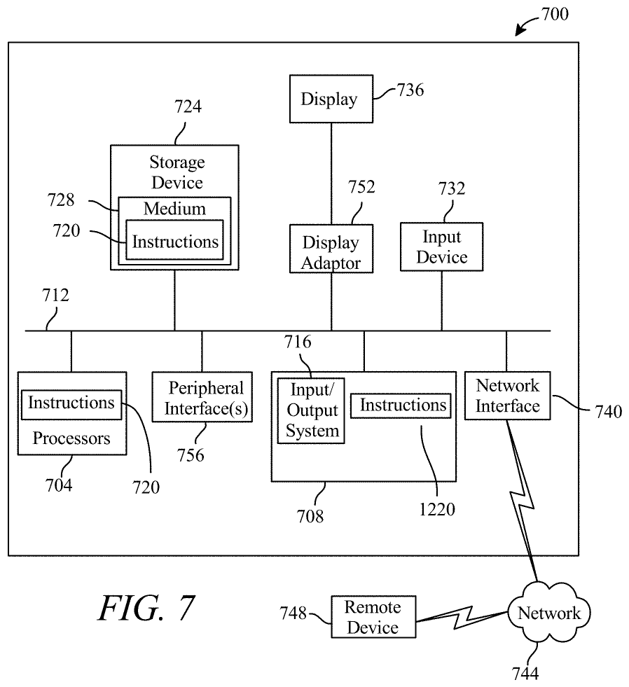

FIG. 7 shows a diagrammatic representation of one embodiment of a computing device in the exemplary form of a computer system 700 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 700 includes a processor 704 and a memory 708 that communicate with each other, and with other components, via a bus 712. Bus 712 may include any of several types of bus structures including, but not limited to, a memory bus, a memory controller, a peripheral bus, a local bus, and any combinations thereof, using any of a variety of bus architectures.

Processor 704 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 704 may be organized according to Von Neumann and/or Harvard architecture as a non-limiting example. Processor 704 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 708 may include various components (e.g., machine-readable media) including, but not limited to, a random-access memory component, a read only component, and any combinations thereof. In one example, a basic input/output system 716 (BIOS), including basic routines that help to transfer information between elements within computer system 700, such as during start-up, may be stored in memory 708. Memory 708 may also include (e.g., stored on one or more machine-readable media) instructions (e.g., software) 720 embodying any one or more of the aspects and/or methodologies of the present disclosure. In another example, memory 708 may further include any number of program modules including, but not limited to, an operating system, one or more application programs, other program modules, program data, and any combinations thereof.

Computer system 700 may also include a storage device 724. Examples of a storage device (e.g., storage device 724) include, but are not limited to, a hard disk drive, a magnetic disk drive, an optical disc drive in combination with an optical medium, a solid-state memory device, and any combinations thereof. Storage device 724 may be connected to bus 712 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 724 (or one or more components thereof) may be removably interfaced with computer system 700 (e.g., via an external port connector (not shown)). Particularly, storage device 724 and an associated machine-readable medium 728 may provide nonvolatile and/or volatile storage of machine-readable instructions, data structures, program modules, and/or other data for computer system 700. In one example, software 720 may reside, completely or partially, within machine-readable medium 728. In another example, software 720 may reside, completely or partially, within processor 704.

Computer system 700 may also include an input device 732. In one example, a user of computer system 700 may enter commands and/or other information into computer system 700 via input device 732. Examples of an input device 732 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 732 may be interfaced to bus 712 via any of a variety of interfaces (not shown) including, but not limited to, a serial interface, a parallel interface, a game port, a USB interface, a FIREWIRE interface, a direct interface to bus 712, and any combinations thereof. Input device 732 may include a touch screen interface that may be a part of or separate from display 736, discussed further below. Input device 732 may be utilized as a user selection device for selecting one or more graphical representations in a graphical interface as described above.

A user may also input commands and/or other information to computer system 700 via storage device 724 (e.g., a removable disk drive, a flash drive, etc.) and/or network interface device 740. A network interface device, such as network interface device 740, may be utilized for connecting computer system 700 to one or more of a variety of networks, such as network 744, and one or more remote devices 748 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 744, may employ a wired and/or a wireless mode of communication. In general, any network topology may be used. Information (e.g., data, software 720, etc.) may be communicated to and/or from computer system 700 via network interface device 740.

Computer system 700 may further include a video display adapter 752 for communicating a displayable image to a display device, such as display device 736. Examples of a display device include, but are not limited to, a liquid crystal display (LCD), a cathode ray tube (CRT), a plasma display, a light emitting diode (LED) display, and any combinations thereof. Display adapter 752 and display device 736 may be utilized in combination with processor 704 to provide graphical representations of aspects of the present disclosure. In addition to a display device, computer system 700 may include one or more other peripheral output devices including, but not limited to, an audio speaker, a printer, and any combinations thereof. Such peripheral output devices may be connected to bus 712 via a peripheral interface 756. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for inspirate sensing to determine a probability of an emergent physiological state comprising:
a fluid channel configured to be in fluidic communication with at least an inspirate;
an inhalation sensor module, in fluidic communication with the fluidic channel, configured to sense and transmit a plurality of inhalation parameters as a function of the at least an inspirate;
an environmental sensor module, in sensed communication with an environment substantially outside of the fluid channel, configured to sense and transmit a plurality of environmental parameters as a function of the environment; and
a processor, in communication with the inhalation sensor module and the environmental sensor module, wherein the processor is further configured to:
receive the plurality of inhalation parameters and the plurality of environmental parameters;
generate a probability of an emergent physiological state, wherein generating a probability of an emergent physiological state further comprises:
inputting at least an environmental parameter of the plurality of environmental parameters and at least an inhalation parameter of the plurality of inhalation parameters to a probabilistic machine learning model; and
generating the probability of an emergent physiological state as a function of the machine learning model.

2. The system of claim 1 wherein the inhalation sensor module further comprises:
at least a gas concentration sensor, configured to sense and transmit at least an inspirate gas concentration parameter as a function of a gas concentration within the at least an inspirate; and
at least an inspirate pressure sensor, configured to sense and transmit at least an inspirate pressure parameter as a function of a pressure of the at least an inspirate.

3. The system of claim 2 wherein the at least a gas concentration sensor comprises:
a light source configured to illuminate a portion of the at least an inspirate; and
a light detector configured to detect a light, wherein the light is either originated from the light source or excited by light from the light source.

4. The system of claim 1 wherein the environmental sensor module further comprises:
at least a cabin pressure sensor, configured to sense and transmit at least a cabin pressure parameter as a function of a pressure of the environment; and
at least a positional sensor, configured to sense and transmit at least a movement parameter as a function of a movement.

5. The system 1 wherein the processor is further configured to:
classify the probability of an emergent physiological state to an intervention, wherein classifying further comprises:
inputting the probability of an emergent physiological state to a classifier; and
classifying the probability of an emergent physiological state to the intervention as a function of the classifier.

6. The system of claim 5, wherein the processor is further configured to:
train the classifier, wherein training the classifier further comprises:
inputting training data to a machine learning algorithm, wherein the training data comprises a plurality of parameters; and
training the classifier as a function of the machine learning algorithm.

7. The system of claim 5, further comprising:
a user-signaling device communicative with the processor and configured to transmit a signal to a user as a function of the intervention.

8. The system of claim 5, wherein the processor is further configured to:
generate at least a confidence metric, wherein generating the at least a confidence metric further comprises:
inputting the intervention class to a machine learning model; and
generating the at least a confidence metric as a function of the machine learning model.

9. The system of claim 1, wherein the processor is further configured to:
train the probabilistic machine learning model, wherein training the probabilistic machine learning model further comprises:
inputting training data to a machine learning algorithm, wherein the training data comprises a plurality of parameters correlated to a probabilistic outcome; and
training the probabilistic machine learning model as a function of the machine learning algorithm.

10. The system of claim 1, wherein the processor is further configured to:
determine a respiration rate as a function of the plurality of inhalation parameters; and
determine a respiration volumetric flow rate as a function of the plurality of inhalation parameters.

11. A method of inspirate sensing to determine a probability of an emergent physiological state comprising:
fluidically communicating, using a fluid channel, at least an inspirate;
sensing and transmitting, using an inhalation sensor module, a plurality of inhalation parameters as a function of the at least an inspirate;
sensing and transmitting, using an environmental sensor module in sensed communication with an environment, a plurality of environmental parameters as a function of the environment;
receiving, using a processor in communication with the inhalation sensor module and the environmental sensor module, the plurality of inhalation parameters and the plurality of environmental parameters;
generating, using the processor, a probability of an emergent physiological state, wherein generating a probability of an emergent physiological state further comprises:
inputting at least an environmental parameter of the plurality of environmental parameters and at least an inhalation parameter of the plurality of inhalation parameters to a probabilistic machine learning model; and
generating the probability of an emergent physiological state as a function of the machine learning model.

12. The method of claim 11 wherein sensing and transmitting the plurality of inhalation parameters further comprises:
sensing and transmitting, using at least a gas concentration sensor, at least an inspirate gas concentration parameter as a function of a gas concentration within the at least an inspirate; and
sensing and transmitting, using at least an inspirate pressure sensor, at least an inspirate pressure parameter as a function of a pressure of the at least an inspirate.

13. The method of claim 12 wherein sensing and transmitting the at least an inspirate gas concentration parameter further comprises:
illuminating, using a light source, a portion of the at least an inspirate; and
detecting, using a light detector, a light, wherein the light is either originated from the light source or excited by light from the light source.

14. The method of claim 11 wherein the sensing and transmitting the plurality of environmental parameters further comprises:
sensing and transmitting, using at least a cabin pressure sensor, at least a cabin pressure parameter as a function of a pressure of the environment; and
sensing and transmitting, using at least a positional sensor, at least a movement parameter as a function of a movement.

15. The method of claim 11 further comprising:
classifying, using the processor, the probability of an emergent physiological state to an intervention, wherein classifying further comprises:
inputting the probability of an emergent physiological state to a classifier; and
classifying the probability of an emergent physiological state to the intervention as a function of the classifier.

16. The method of claim 15, further comprising:
transmitting, using a user-signaling device communicative with the processor, a signal to a user as a function of the intervention.

17. The method of claim 15, further comprising:
generating, using the processor, at least a confidence metric, wherein generating the at least a confidence metric further comprises:
inputting the intervention class to a machine learning model; and
generating the at least a confidence metric as a function of the machine learning model.

18. The method of claim 15, further comprising:
training, using the processor, the classifier, wherein training the classifier further comprises:
inputting training data to a machine learning algorithm, wherein the training data comprises a plurality of parameters; and
training the classifier as a function of the machine learning algorithm.

19. The method of claim 11, further comprising:
training, using the processor, the probabilistic machine learning model, wherein training the probabilistic machine learning model further comprises:
inputting training data to a machine learning algorithm, wherein the training data comprises a plurality of parameters correlated to a probabilistic outcome; and
training the probabilistic machine learning model as a function of the machine learning algorithm.

20. The method of claim 1, further comprising:
determining, using the processor, a respiration rate as a function of the plurality of inhalation parameters; and
determining, using the processor, a respiration volumetric flow rate as a function of the plurality of inhalation parameters.
